(19) United States
(12) Patent Application Publication
(10) Pub. No.: US 2026/0188425 A1
(43) Pub. Date: Jul. 2, 2026

(54) Method Of Extracting Information About Protein Sequence Modifications

(71) Applicant: HOFFMANN-LA ROCHE INC., Little Falls, NJ (US)

(72) Inventors: Marc Alexander BUETTNER, Penzberg (DE); Juergen FICHTL, Penzberg (DE); Eva VOSIKA, Penzberg (DE)

(73) Assignee: HOFFMANN-LA ROCHE INC., Little Falls, NJ (US)

(21) Appl. No.: 18/751,160

(22) PCT Filed: Dec. 23, 2022
(86) PCT No.: PCT/EP2022/087710

§ 371 Date: Jun. 21, 2024

(30) Foreign Application Priority Data
Dec. 23, 2021 (EP) 212174379

(51) Int. Cl. G16B 20/20; B01D 15/08; G01N 30/02; G01N 30/72; G01N 30/88; G01N 33/68; H01J 49/00

(52) U.S. Cl. CPC G16B 20/20; B01D 15/08; G01N 30/7233; G01N 30/88; G01N 33/6848; H01J 49/004; G01N 2030/027; G01N 2030/8831

(57) ABSTRACT

Methods of extracting information about protein sequence modifications in a protein are disclosed. Protein data derived from mass spectrometry measurements performed on peptides from at least two enzymatic digests is received. Candidate sequence modifications are identified. A subset of the candidate sequence modifications that have a higher average probability of representing true sequence modifications than the rest of the candidate sequence modifications are determined. The determination of the subset of candidate sequence modifications comprises a step of selecting candidate sequence modifications dependent on the candidate sequence modifications being at amino acid sequence positions that are covered by at least two different peptide species that each contain the modification.

The present disclosure relates to extracting information about protein sequence modifications in a protein.

Complex biotechnological manufacturing processes can introduce various modifications in therapeutic proteins, potentially resulting in highly heterogeneous products. Depending on their positions and types, these modifications can significantly influence structure, stability, immunogenicity and biological activity of the protein. Thus, an extensive characterization of therapeutic proteins is fundamental to providing patients with safe and efficacious medicines.

A frequently used technique for identifying modifications in therapeutic proteins is proteolytic digestion of the protein combined with chromatographic peptide separation and mass spectrometry (LC-MS/MS). The proteolytic enzyme Trypsin is the gold standard for this approach. Other proteases such as chymotrypsin, LysC, LysN, AspN, GluC and ArgC are also used in proteomics but to a lesser extent. Multi-enzyme strategies have been proposed to maximize sequence coverage through the utilization of a combination of parallel or sequential proteolytic digests.

These approaches lead to large amounts of mass spectrometry (MS) data. This is especially true when looking for the presence of sequence variants (SVs). SVs represent amino acid substitutions in the primary structure of proteins, which can occur through mutations and misincorporations. In order to identify SVs in MS raw data, special software like Mascot Error Tolerance Search (Matrix Science Inc.) or Byonic (Protein Metrics Inc.) may be employed. These software solutions can identify unexpected mass shifts and annotate these mass shifts as modifications or sequence variants.

Since there are numerous possibilities for SVs on each amino acid within the peptide sequence, the chances of random matches identified by the software MS/MS algorithms (“false positive hits”) are high compared with a regular database search for post-translational modifications (PTMs) like chemical modifications or glycans.

Distinguishing true positives from a large number of false positives is challenging. This verification process is currently performed manually and can take several days or even weeks to accomplish, as well as being prone to human error.

For a typical sequence variant analysis experiment, sample preparation, sample analysis on LC-MS/MS instruments and software search takes approximately 2-3 days. However, the subsequent manual “hit verification” by checking various criteria, such as retention time, mass accuracy, and the MS/MS spectra may take days or even weeks.

It is an object of the invention to provide improved methods for extracting information about protein sequence modifications such as SVs.

According to an aspect of the invention, there is provided a computer-implemented method of extracting information about protein sequence modifications in a protein, comprising: receiving protein data derived at least partially from mass spectrometry measurements performed on peptides obtained by at least two different enzymatic digests performed on respective sub-samples of a representative sample of a protein; identifying candidate sequence modifications in the protein using the received protein data; determining a subset of the candidate sequence modifications that have a higher average probability of representing true sequence modifications than the rest of the candidate sequence modifications; and outputting data representing the determined subset of candidate sequence modifications, wherein: the determination of the subset of candidate sequence modifications comprises a step of selecting candidate sequence modifications dependent on the candidate sequence modifications being at amino acid sequence positions that are covered by at least two different peptide species that each contain the modification.

Thus, a method is provided that identifies a subset of candidate sequence modifications that are more likely to be correct via a computer automated procedure, thereby saving human effort/time and/or reducing errors. Receiving protein data from peptides obtained by at least two different enzymatic digests increases reliable coverage of the protein sequence and, as explained below, provides the basis for further filtering criteria that can reduce false positives further.

In an embodiment, the determination of the subset of candidate sequence modifications comprises a step of selecting candidate sequence modifications dependent on the candidate sequence modifications being at amino acid sequence positions where a ratio of the number of peptide species covering the amino acid sequence position and containing the candidate sequence modification to the number of peptide species covering the amino acid sequence position and not containing the candidate sequence modification is equal to or higher than a predetermined ratio threshold. This criterion excludes candidate modifications in a peptide species where the corresponding modified peptide species occurs relatively rarely in comparison with the corresponding unmodified (wild type) peptide species. The inventors have found this filtering approach to be effective in reducing false positives.

In an embodiment, the method includes pre-processing the protein data to identify data relating to a selected subset of peptide species and excluding the selected subset of peptide species from use in the determination of the subset of candidate sequence modifications. The pre-processing further improves performance.

The pre-processing may comprise excluding peptide species for which a) a candidate modification is present; and b) a highest intensity in the mass spectrometry measurements of a corresponding peptide species with the same amino acid sequence but without the candidate modification (“wild type”) is below a predetermined intensity threshold. This filtering approach is based on the realisation that some peptides are less intense than others due to their physicochemical characteristics, which are defined by their respective sequences. Modifications to such sequences will not typically change the ionization characteristics completely. Thus, a low intensity “wild type” will tend to be associated with low intensity (and therefore relatively unreliably identified) modified peptides. Excluding such peptide species from subsequent analysis therefore contributes efficiently to reducing false positives.

The pre-processing may comprise excluding peptide species for which a) a candidate modification is present; and b) a highest accuracy score in the mass spectrometry measurements of a corresponding peptide species with the same amino acid sequence but without the candidate modification is below a predetermined score threshold. The accuracy score represents a degree of matching between theoretical and observed fragments in the mass spectrometry measurements. This filtering approach is based on the realisation that a low accuracy score for a wild type peptide species indicates that information obtained from corresponding peptide species with a modification will be relatively unreliable. Excluding such peptide species from subsequent analysis therefore contributes efficiently to reducing false positives.

The pre-processing may comprise excluding each peptide species having a candidate modification at a cleavage site for the enzymatic digest that produced the peptide species. This filtering approach is based on the realisation that modifications can influence the digest behaviour of an enzyme, which means that peptide species having a start or end point that corresponds with the position of a modification are not optimal for detecting that modification. Excluding these peptide species from subsequent analysis therefore contributes to reducing false positives.

The pre-processing may comprise excluding peptide species having a length above a predetermined length threshold. This filtering approach is based on the realisation that modifications identified in long peptide species are generally more difficult to verify and therefore less reliable. Excluding peptide species that are longer than the predetermined length threshold is therefore effective for reducing false positives.

In an embodiment, the determination of the subset of candidate sequence modifications comprises a step of selecting candidate modifications dependent on the candidate sequence modifications being at amino acid sequence positions that are covered by at least two different peptide species that each contain the modification and have been derived from peptides obtained using different enzymatic digests. The inventors have found that this approach is highly effective in removing false positives.

According to a further aspect of the invention, there is provided a computer-implemented method of extracting information about protein sequence modifications in a protein, comprising: receiving protein data derived at least partially from mass spectrometry measurements performed on peptides obtained by at least two different enzymatic digests performed on respective sub-samples of a representative sample of a protein; identifying candidate sequence modifications in the protein using the received protein data; determining a subset of the candidate sequence modifications that have a higher average probability of representing true sequence modifications than the rest of the candidate sequence modifications; and outputting data representing the determined subset of candidate sequence modifications, wherein: the determination of the subset of candidate sequence modifications comprises a step of selecting candidate sequence modifications dependent on the candidate sequence modifications being at amino acid sequence positions where a ratio of the number of peptide species covering the amino acid sequence position and containing the candidate sequence modification to the number of peptide species covering the amino acid sequence position and not containing the candidate sequence modification is equal to or higher than a predetermined ratio threshold.

Thus, a method is provided that identifies a subset of candidate sequence modifications that are more likely to be correct via a computer automated procedure, thereby saving human effort/time and/or reducing errors. Excluding candidate modifications according to the defined ratio excludes candidate modifications where the corresponding modified peptide species occurs relatively rarely in comparison with the corresponding unmodified (wild type) peptide species. The inventors have found this filtering approach to be effective in reducing false positives.

In some embodiments, the received protein data is derived from mass spectrometry measurements performed on peptides obtained by five or six different enzymatic digests performed on respective sub-samples of the representative sample of the protein, preferably wherein each of the five or six enzymatic digests uses a different one of the following: Trypsin, Thermolysin, AspN, Pronase, Pepsin, ProAlanase. The inventors have found that using these specific numbers of digests provides an advantageous balance of high sensitivity to few false positives.

In some embodiments, the method further comprises identifying one or more groups of peptide species in the received protein data, each group of peptide species exclusively containing peptide species that all have the same candidate sequence modification, the candidate sequence modification being in the determined subset of candidate sequence modifications and different for each group; and outputting data representing which peptide species are in each of the identified groups. Identifying groups of peptide species that all have the same candidate sequence modification makes it possible to present information to a user in a more organised manner and facilitates efficient assessment of candidate sequence modifications. The approach helps to avoid duplication of effort by a user assessing the same candidate sequence modification multiple times in different peptide species.

According to a further aspect of the invention, there is provided a computer-implemented method of extracting information about protein sequence modifications in a protein, comprising: receiving protein data derived at least partially from mass spectrometry measurements performed on peptides obtained by at least two different enzymatic digests performed on respective sub-samples of a representative sample of a protein; identifying candidate sequence modifications in the protein using the received protein data; determining a subset of the candidate sequence modifications that have a higher average probability of representing true sequence modifications than the rest of the candidate sequence modifications; and outputting data representing the determined subset of candidate sequence modifications, wherein: the determination of the subset of candidate sequence modifications comprises a step of selecting candidate sequence modifications dependent on the candidate sequence modifications satisfying a quantification condition, the quantification condition indicating that an amount detected by the mass spectrometry measurements of at least a selected subset of peptide species with the candidate sequence modification relative to a total amount detected by the mass spectrometry measurements of the same peptide species with and without the candidate sequence modification is above a predetermined quantification threshold.

Thus, a method is provided that identifies a subset of candidate sequence modifications that are more likely to be correct via a computer automated procedure, thereby saving human effort/time and/or reducing errors. Excluding candidate modifications based on whether a quantification condition is satisfied has been found to be particularly effective for reducing false positives.

In an embodiment, the at least two different enzymatic digests comprise one or more sequence-specific enzymatic digests and one or more non-specific enzymatic digests, and the selected subset of the peptide species is selected to exclude peptide species derived using the one or more non-specific enzymatic digests, at least for candidate sequence modifications that are covered by at least one peptide species derived using a sequence-specific enzymatic digest. Preferentially or exclusively taking into account peptide species from sequence-specific enzymatic digests has been found to provide particularly high performance, allowing further reduction in false positives.

Embodiments of the disclosure will be further described by way of example only with reference to the accompanying drawings.

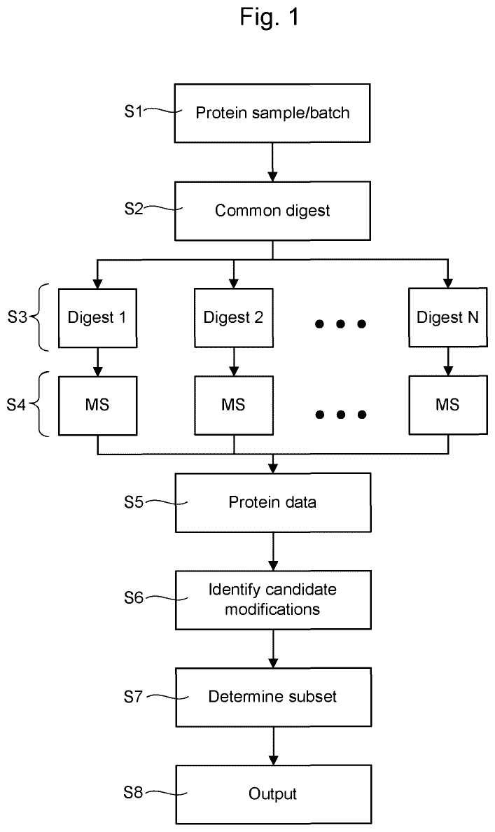

FIG. 1 is a flow chart depicting a method of extracting information about a protein sequence.

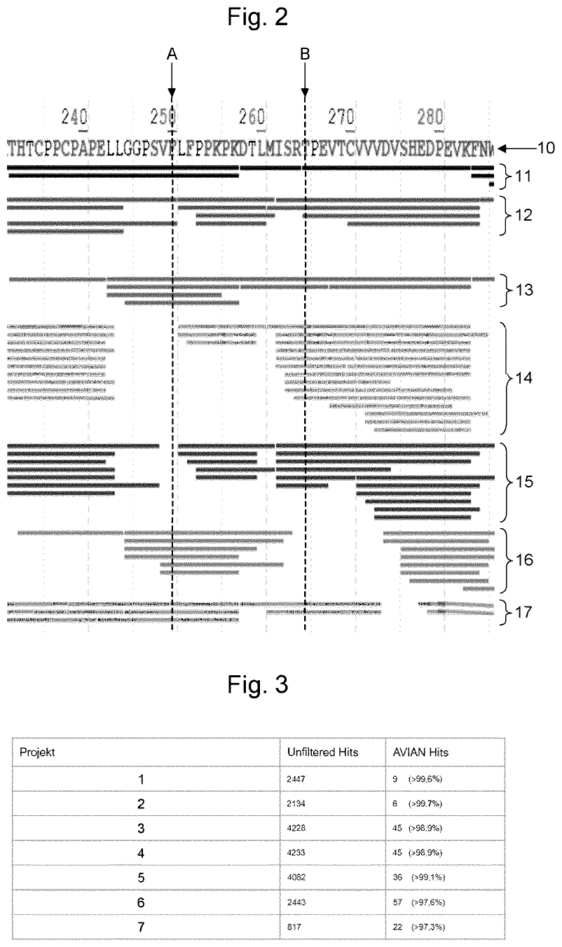

FIG. 2 schematically depicts coverage of a portion of a protein sequence by peptide species from different digests.

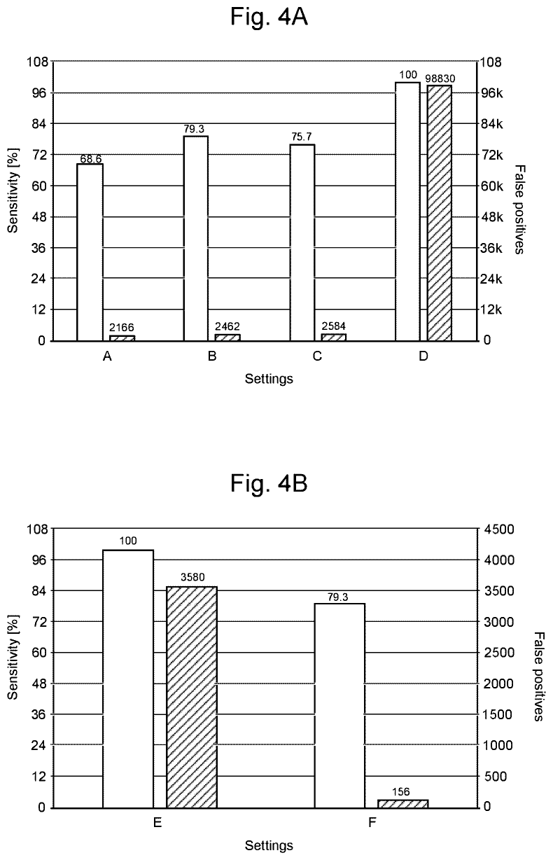

FIGS. 3-8 show data demonstrating performance of methods of the present disclosure. FIG. 3 is based on samples from real product development projects. FIGS. 4-8 are based on artificially generated samples in which known amounts of sequence variations are deliberately “spiked” into the samples.

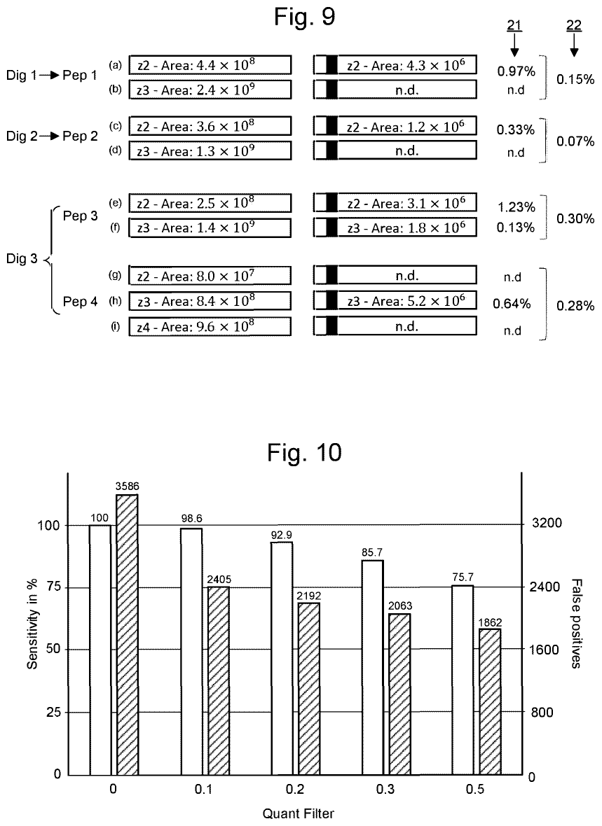

FIG. 9 schematically depicts example detected amounts of peptide species with and without a candidate modification for different digests and charge states.

FIG. 10 shows data demonstrating performance of methods of the present disclosure that use a quantification threshold, the data derived using artificially generated samples in which known amounts of sequence variations are deliberately “spiked” into the samples.

Various embodiments of the disclosure relate to methods that are computer-implemented. Each step of the disclosed methods may be performed by a computer in the most general sense of the term, meaning any device capable of performing the data processing steps of the method, including dedicated digital circuits. The computer may comprise various combinations of known computer elements, including for example CPUs, RAM, SSDs, motherboards, network connections, firmware, software, and/or other elements known in the art that allow the computer to perform the required computing operations. The required computing operations may be defined by one or more computer programs. The one or more computer programs may be provided in the form of media or data carriers, optionally non-transitory media, storing computer readable instructions. When the computer readable instructions are read by the computer, the computer performs the required method steps. The computer may consist of a self-contained unit, such as a general-purpose desktop computer, laptop, tablet, mobile telephone, or other smart device. Alternatively, the computer may consist of a distributed computing system having plural different computers connected to each other via a network such as the internet or an intranet.

Embodiments of the disclosure concern extracting information about protein sequence modifications in a protein. The framework of an example method is depicted schematically in FIG. 1 and described below.

The method starts with the provision of a representative sample of a protein to be analysed (step S1). The protein may be a therapeutic protein for example. The sample may be provided in any of a variety of forms known in the art. For example, a typical protein sample may be obtained directly from the supernatant of a cell culture, be recovered by cell disruption, solubilisation and renaturation of inclusion bodies from bacterial cells or by extraction from a tissue. The sample may also have been subjected to further mechanical or chemical purification steps, such as filtration, diafiltration, dialysis, centrifugation, precipitation or chromatography. In one aspect, the protein sample is essentially free from other proteins, that is, it contains less than 20%, optionally less than 10%, optionally less than 5%, optionally less than 2%, optionally less than 1%, optionally less than 0.5%, optionally less than 0.2% of other proteins. Typically, around 300-500 μg of sample may be provided.

The sample may be processed initially as a single sample. For example, the sample may be subjected to a common digest (step S2), such as enzymatic deglycosylation using PNGase. It may be desirable to remove glycans because they would lead to further fragments that would make interpretation of the peptide patterns more difficult.

In step S3, the representative sample is split into sub-samples and each sub-sample is subjected to a different enzymatic digest. Each digest will use a different enzyme or a different combination of enzymes. Other conditions may also vary between different digests, such as the time for which the digestion process is allowed to proceed before being stopped. Typically, each digest will use a single enzyme, but using a combination of enzymes in a single sub-sample may sometimes be appropriate (e.g. to obtain peptides of suitable length for subsequent mass spectrometry steps). In arrangements of the present disclosure, at least two different enzymatic digests are used (on two corresponding sub-samples). In some arrangements, the number of enzymatic digests is higher, for example at least three, optionally at least four, optionally at least five, optionally at least six, optionally at least seven, optionally at least eight, optionally at least nine. In one arrangement, the number of enzymatic digests is from 5 to 9, preferably 5 or 6. Processing of the sub-samples by the different digests is depicted schematically in FIG. 1 by boxes labelled “Digest 1”, “Digest 2”, etc. In principle, any number N of such digests may be performed.

Each enzymatic digest uses a different one or combination of the following: Trypsin; Thermolysin; AspN; Elastase; Chymotrypsin; LysC; LysN; GluC; ArgC; Pronase; Pepsin; ProAlanase. In one particular embodiment, all of the following nine digests are used: Trypsin only; Thermolysin only; AspN only; Elastase only; Chymotrypsin only; a combination of LysC+GluC (in the ratio of 1:20 for example); Pronase only; Pepsin only; ProAlanase only. The digests may be allowed to proceed for between 0.5 hours and 4 hours, for example, depending on the enzymes used.

The enzymes used in each digest belong to the class of proteases and drive proteolysis, which is the breakdown of proteins into smaller polypeptides by cleaving of peptide bonds. Locations of the cleaving will depend on the protein that is being digested and on the enzyme or combination of enzymes that are present. Each digest will thus produce a different population of peptide species.

In step S4, the peptides obtained from the digests are processed by mass spectrometry. The outputs from individual digests may be processed separately from each other or in combination. In the example shown in FIG. 1, the output from Digest 1 (peptides obtained by applying Digest 1 to a sub-sample) is processed in a first mass spectrometry process MS, the output from Digest 2 (the peptides obtained by applying Digest 2 to a sub-sample) is processed in second mass spectrometry process MS, etc. In some arrangements, each mass spectrometry process comprises liquid chromatography-tandem mass spectrometry (LC-MS/MS). LC-MS/MS is a well-known analytical chemistry technique for analysing peptide species. A liquid chromatography column is coupled to an ion source of a mass spectrometry system, which allows components of a sample separated by the liquid chromatography to be fed directly into the mass spectrometry system. In order to obtain sequence information corresponding to peptide m/z signals, the mass spectrometry system is operated in tandem mode (MS/MS) to achieve extended information about sample composition. Components received from the liquid chromatography column are ionized and subsequently separated according to their mass-to-charge ratio in a first mass analyzer. The separated ions are then split into smaller fragment ions, which may be referred to as peptide fragments. The peptide fragments are separated in a second mass analyzer run (e.g., in a second mass spectrometry step, either in the same or a different mass analyser) and detected.

Steps S5 onwards may be computer-implemented.

In the computer-implemented steps, each peptide species may be defined by at least the following: i) an amino acid sequence; ii) the enzymatic digest that produced the peptide species; and iii) a modification status indicating whether a candidate modification is present and, if a candidate modification is present, the nature and amino acid sequence position of the modification. In some arrangements, each peptide species is further defined by iv) a charge state in the mass spectrometry measurements.

In step S5, protein data derived at least partially from the mass spectrometry measurements of step S4 are received. The protein data is thus derived from mass spectrometry measurements performed on peptides obtained by different enzymatic digests applied to respective sub-samples. The protein data may take any of various forms known in the art for representing information about peptides analysed in this way. For example, the protein data may comprise information obtained by comparing the masses of measured peptides (MS) or peptide fragments (MS/MS) with predicted theoretical values for corresponding peptide species with and without modifications. Special software like Mascot Error Tolerance Search (Matrix Science Inc.) or Byonic (Protein Metrics Inc.) may be employed. These software solutions can identify unexpected mass shifts and annotate these mass shifts as sequence modifications.

In step S6, the protein data received in step S5 is used to identify candidate sequence modifications in the protein. This may be achieved by analysing the protein data to identify unexpected mass shifts as being candidate sequence modifications or the protein data may already have been annotated with this information, as mentioned above. The resulting candidate sequence modifications would normally need to be reviewed manually to check for plausibility (i.e. to reduce the number of false positives). The method steps described below replace the manual review with an automatic review, or greatly reduce the number of candidate modifications that need to be reviewed manually.

In step S7, a subset of the candidate sequence modifications that have a higher average probability of representing true sequence modifications than the rest of the candidate sequence modifications is determined. The determined subset represents a list of modifications that are thus likely to be true modifications. The list is obtained through computer-implemented steps, thus reducing or avoiding the need for manual review. The list may be output (step S8) to a user according to user preferences (e.g. as an output data stream or file, or as an indication on a computer display). The determination of the subset of candidate sequence modifications includes filtering based on coverage of amino acid positions by peptides species (of the peptides derived from the digests), optionally based on coverage by peptide species from different enzymatic digests.

Coverage of amino acid positions for an example segment of a protein sequence is depicted schematically in FIG. 2. Here horizontal lines below the sequence (labelled 10) represent different peptide species. The peptide species are grouped into groups 11-17 according to which enzymatic digest was used to obtain them. Thus, group 11 shows portions of peptide species obtained using a first enzymatic digest, group 12 shows portions of peptide species obtained using a second enzymatic digest, group 13 shows portions of peptide species obtained using a third enzymatic digest, etc. As can be seen, coverage by the different peptide species varies according to position along the amino acid sequence 10. At position A, for example, coverage is provided by peptide species in groups 11, 12, 13, 16 and 17 (with no coverage from groups 14 and 15), while at position B, coverage is provided by peptide species in groups 11-15 and 17 (with no coverage from group 16).

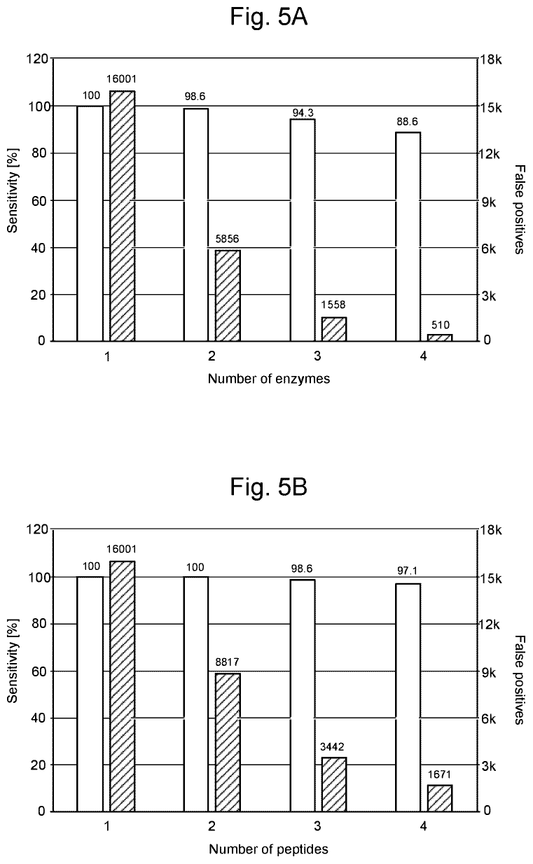

In an arrangement, the determination of the subset in step S7 comprises selecting candidate sequence modifications dependent on the candidate sequence modifications being at amino acid sequence positions that are covered by at least two different peptide species (derived from peptides obtained by the at least two enzymatic digests) that each contain the modification. Thus, candidate sequence modifications at positions that are not covered by a high enough number of different peptide species with the modification (from the same or from different digests) are excluded. The determination step S7 thus acts as a filter to exclude candidate modifications based at least on how the corresponding sequence position is covered by peptide species. A filter setting corresponding to this type of filtering is referred to in the examples below as “noPep”. Observation of the same modification in many different peptide species indicates a higher likelihood of the modification being a true modification. Excluding modifications that are only present in a single peptide species (or small number of different peptide species) thus reduces false positives (i.e. candidate sequence modifications that are untrue) efficiently. Efficacy of this filtering approach is demonstrated by the data shown in FIGS. 4 and 5 described below. As described below, step S7 may additionally include filters based on other exclusion criteria.

The filtering based on coverage by different peptide species may be made stricter, thereby increasing the exclusion of false positives. An optimum balance may be made between reliably excluding false positives and avoiding or minimizing exclusion of true positives. In some arrangements, the filtering is strengthened to exclude candidate sequence modifications that are not covered by at least three, optionally at least four, optionally at least five, optionally at least six, different peptide species having the modification. The effects of such strengthened filtering are shown in FIG. 5B.

In some embodiments, the determination of the subset in step S7 comprises selecting candidate sequence modifications dependent on the candidate sequence modifications being at amino acid sequence positions that are covered by at least two different peptide species that each contain the modification and have been derived from peptides obtained using different enzymatic digests. Thus, candidate sequence modifications at positions that are not covered by peptides species from at least two different enzymatic digests are excluded. Determination step S7 thus acts as a filter to exclude candidate modifications based at least on how the corresponding sequence position is covered by peptides species from different enzymatic digests. A filter setting corresponding to this type of filtering is referred to in the examples below as “No_of_digests”. This approach is highly effective in removing false positives because such false positives occur mainly at random and are unlikely to be present in peptide species produced by multiple different digests. True positives, on the other hand, will be seen in peptide species produced by different enzymes. Efficacy of this filtering approach is demonstrated by the data shown in FIG. 3 described below.

The filtering based on coverage by different enzymatic digests may be made stricter, thereby increasing the exclusion of false positives. An optimum balance may be made between reliably excluding false positives and avoiding or minimizing exclusion of true positives. In some arrangements, the filtering is strengthened to exclude candidate sequence modifications that are not covered by peptides species having the modification from at least three, optionally at least four, optionally at least five different enzymatic digests. The effects of such strengthened filtering are shown in FIG. 5A.

### Pre-Processing Before Step S7

In some arrangements, the protein data is pre-processed before the analysis of step S7. The pre-processing of the protein data may comprise identifying data relating to a selected subset of peptide species and excluding the selected subset of peptide species from use in the determination of the subset of candidate sequence modifications in step S7.

In some arrangements, peptide species are excluded where a) a candidate modification is present; and b) a highest intensity (peak height at apex) in the mass spectrometry measurements of a corresponding peptide species with the same amino acid sequence but without the candidate modification is below a predetermined intensity threshold. A filter setting corresponding to this type of filtering is referred to in the examples below as “WT Intensity”. A low intensity in the mass spectrometry measurements for such a “wild type” peptide species (i.e. a peptide species without the modification) indicates that information obtained from corresponding peptide species with a modification will be relatively unreliable. In essence, the mass spectrometry measurements are relatively inefficient at measuring peptide species having this particular sequence (with or without the modification). In other words, some peptides are less intense than others due to their physicochemical characteristics, which are defined by their respective sequences. Modifications to such sequences will not typically change the ionization characteristics completely. Thus, a low intensity “wild type” will tend to be associated with low intensity (and therefore relatively unreliable) modified peptides. Excluding such peptide species from subsequent analysis therefore contributes efficiently to reducing false positives. This is demonstrated by the data shown in FIG. 7 discussed below.

In some arrangements, peptide species are excluded where a) a candidate modification is present; and b) a highest accuracy score in the mass spectrometry measurements of a corresponding peptide species with the same amino acid sequence but without the candidate modification is below a predetermined score threshold. A filter setting corresponding to this type of filtering is referred to in the examples below as “WT Score”. An “accuracy score” in this context refers to the result of applying an algorithm that compares a theoretical fragmentation pattern with a fragmentation pattern produced by the mass spectrometry measurements. The accuracy score may be a metric that quantifies a degree of matching/correlation between theoretical and observed fragments. A higher score indicates higher correlation (better matching). In a similar manner to the case for low intensities discussed above, a low accuracy score for a wild type peptide species indicates that information obtained from corresponding peptide species with a modification will be relatively unreliable. Excluding such peptide species from subsequent analysis therefore contributes efficiently to reducing false positives.

In some arrangements, the pre-processing comprises excluding each peptide species having a candidate modification at a cleavage site for the enzyme that produced the peptide species. A filter setting corresponding to this type of filtering is referred to in the examples below as “Exclude Cleavage Site”. Modifications can influence the digest behaviour of an enzyme, which means that peptide species having a start or end point that corresponds with the position of a modification are not optimal for detecting that modification. Excluding these peptide species from subsequent analysis therefore contributes to reducing false positives.

In some arrangements, the pre-processing comprises excluding peptide species having a length above a predetermined length threshold. A filter setting corresponding to this type of filtering is referred to in the examples below as “PeptideLength”. Modifications identified in long peptide species are generally more difficult to verify and therefore less reliable. Long peptide species (e.g. >3500 Da) are typically highly charged (4-6). Highly charged peptides typically cause poor MS/MS coverage. The reason is that highly charged peptides accelerate much faster into the collision cell of the mass spectrometry apparatus, which leads to fewer fragments compared to a lower charged peptide species. It is possible to use different fragmentation modes to get “good” MS/MS data (i.e. high fragment ion coverage, high intensity) from highly charged peptides. However, such fragmentation modes (e.g., Electron-Transfer Dissociation (ETD)) tend to result in lower MS/MS scores than alternatives (e.g. Collision Induced Dissociation (CID)) for smaller peptide species with typically fewer charges. Excluding peptide species that are longer than the predetermined length threshold is therefore effective for reducing false positives.

### Other Selection Criteria for Step S7

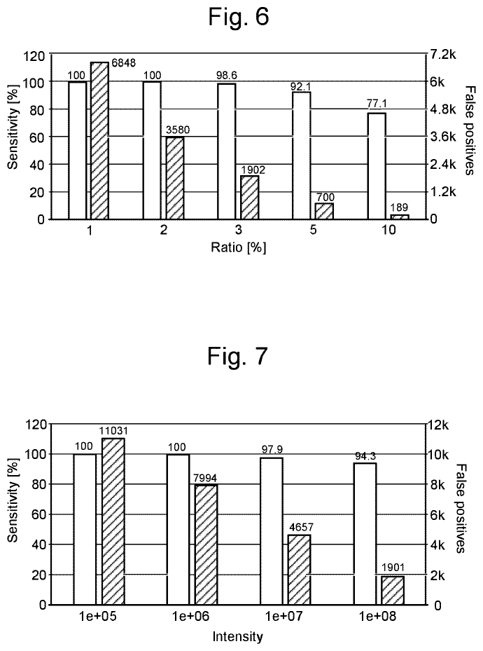

In some arrangements, the determination of the subset of candidate sequence modifications in step S7 comprises a step of selecting candidate sequence modifications dependent on the candidate sequence modifications being at amino acid sequence positions where a ratio of the number of peptide species covering the amino acid sequence position and containing the candidate sequence modification to the number of peptide species covering the amino acid sequence position and not containing the candidate sequence modification is equal to or higher than a predetermined ratio threshold. A filter setting corresponding to this type of filtering is referred to in the examples below as “Ratio Filter”. This criterion excludes candidate modifications in a peptide species where the corresponding modified peptide species occurs relatively rarely in comparison with the corresponding unmodified (wild type) peptide species. Efficacy of this filtering approach is demonstrated by the data shown in FIG. 6 described below. In some embodiments, the minimum ratio (predetermined ratio threshold) is set in the range 2-10%, preferably in the range 2-5%, preferably in the range 2-4%, preferably in the range 2.5-3.5%, preferably about 3%.

### Experiments Demonstrating Performance

Experiments demonstrating performance are described below with reference to “filter settings” which define configuration options for selecting or discounting candidate sequence modifications to obtain the subset of candidate sequence modifications that is output by the method. The filter settings include the following (some of which have been discussed above).

“SV Score”—an accuracy score representing a degree of correlation between theoretical and observed fragments (peptide species) in the mass spectrometry measurements for fragments containing the candidate modification.

“WT Score”—an accuracy score representing a degree of correlation between theoretical and observed fragments (peptide species) in the mass spectrometry measurements for fragments not containing the candidate modification.

“ppm”—the mass accuracy of the mass spectrometry measurements.

“MS1 Corr”—a metric representing the highest MS1 correlation (comparing the theoretical and measured isotope pattern) for all identifications of a peptide species with the same sequence, digest type, charge state, modification, and modification position. A score of 1 is a perfect match.

“PeptideLength”—the number of amino acids in the peptide species.

“WT Intensity”—indicates the highest intensity (peak height at apex) in the mass spectrometry measurements of all identifications of an unmodified peptide species corresponding to the modified peptide species containing the candidate modification, with the same digestion type and taking into account all present charge states.

“Exclude Cleavage Site”—a “yes” or “no” setting indicating whether candidate sequence modifications corresponding to enzyme cleavage sites are excluded.

“Ratio Filter”—a ratio of the number of peptide species covering the amino acid sequence position and containing the candidate sequence modification to the number of peptide species covering the amino acid sequence position and not containing the candidate sequence modification.

“noPep”—the number of peptide species that cover the amino acid sequence position of the candidate sequence modification and contain the modification.

“No_of_digests”—the number of peptide species that cover the amino acid sequence position of the candidate sequence modification, contain the modification, and have been derived from peptides obtained using different enzymatic digests.

“XIC Ratio”—indicates minimal XIC Ratio of the candidate sequence modifications, using the Ratio of the XIC-Area of the candidate sequence modification to the XIC-Area of the corresponding peptides species not containing the modification.

FIG. 3 is a table depicting application of the method to seven different product development projects.

To generate the data shown in FIG. 3, purified protein samples (usually 350 μg) from seven different projects were denatured in 8 M Guanidine hydrochloride at pH 7.0 and reduced by adding DTT (dithiothreitol) and incubation for 1 h at 37° C. 8-carboxymethylation of the reduced samples was performed by adding iodoacetic acid. Prior to digestion with several enzymes, the buffer was exchanged to digestion buffer (50 mM Tris, 2 mM CaCl2, pH 7.5) using NAP5 columns. The samples were split into nine equal fractions to add different enzymes. The digestion conditions were enzyme dependent. The following conditions were used:

TABLE 1
Reaction conditions for enzymatic digests

| | Enzyme/ | | |
|---|---|---|---|
| | Substrate | Digest | |
| Enzyme | Ratio | duration | Quench |
| Trypsin | 1:20 | 1 h | by adding 10% TFA |
| Thermolysin | 1:67 | 1 h | by adding EDTA |
| AspN | 1:70 | 4 h | by adding 10% TFA |
| Elastase | 1:70 | 4 h | by adding 10% TFA |
| Chymotrypsin | 1:20 | 1 h | by adding 10% TFA |
| LysC + GluC | 1:20 each | 1 h | by adding 10% TFA |
| Pronase | 1:500 | 1 h | by adding 10% TFA |
| Pepsin | 1:25 | 1 h | by heating to 95° C. |
| Pro-Alanase | 1:25 | 1 h | by heating to 95° C. |

The resulting digests were resolved by RP-LC coupled to an Orbitrap Fusion mass spectrometer from Thermo Fisher Scientific (data dependent setup). For separation, a 120-minute gradient (mobile phase A: Water with 0.1% v/v formic acid (FA); mobile phase B: Acetonitrile with 0.1% v/v FA) on a ACQUITY UPLC CSH C18 Column (Waters, 130 Å, 1.7 μm, 2.1 mm×150 mm) was used. For the Fusion-Orbitrap a data dependent setup was used.

The first column (Projekt) of FIG. 3 contains a number representing the project. The second column represents the number of candidate sequence modifications identified in step S6 of the method. The third column shows the number of sequence modifications in the subset determined in step S7. The number in brackets indicates reduction rate of false positive hits; e.g., first row 9/2447=0.4% means 99.6% of hits get filtered out. Filter settings were as follows: WT Intensity >1e6; SV Score >140; WT Score >220; PeptideLength: 5-32; MS1 Corr >0.95; ppm <4; No_of_digests >=2 (data applied by Orbitrap Fusion LC-MS/MS data dependent top time; CID in IonTrap; 120 min gradient, 9 digests). These results demonstrate that the method is capable of identifying true positives with high reliability from a large number of candidate sequence modifications, as well as reliably excluding all or substantially all of the false positives from amongst such a large number of candidate sequence modifications. It is noted that the data of FIG. 3 is not an artificially created data set with spiked-in sequence modifications. The data in FIG. 3 are derived from analysis of samples from actual projects using the method. Sensitivity (i.e. true-positives) results cannot be provided in this case, since it is not known what SVs are actually present in a sample. This is in contrast to the spiked-in data underlying FIGS. 4-8 (discussed below) where it is possible to evaluate how many of the known and expected SVs in the sample have been identified using the method.

FIGS. 4-8 and 10 are bar-chart graphs showing the results of experiments to demonstrate performance of embodiments of the present disclosure.

In order to determine in a quantitative manner whether all sequence modifications present in a sample can be detected, nine different test samples (1-9), each containing a first antibody, were spiked by adding a second antibody into the solution at 0.5% levels, as shown in Table 2. An additional set of nine test samples (10-18) was generated by spiking the second antibody with the first antibody at a level of 0.5%.

TABLE 2
Spiking samples

| | | | Ratio 1st to | Ratio 1st to |
|---|---|---|---|---|
| | 1st | 2nd | 2nd antibody in | 2nd antibody in |
| | antibody | antibody | Samples 1-9 | Samples 10-18 |
| 1 | A | J | 99.5% and 0.5% | 0.5% and 99.5% |
| 2 | B | J | 99.5% and 0.5% | 0.5% and 99.5% |
| 3 | C | K | 99.5% and 0.5% | 0.5% and 99.5% |
| 4 | D | J | 99.5% and 0.5% | 0.5% and 99.5% |
| 5 | E | L | 99.5% and 0.5% | 0.5% and 99.5% |
| 6 | E | J | 99.5% and 0.5% | 0.5% and 99.5% |
| 7 | F | J | 99.5% and 0.5% | 0.5% and 99.5% |
| 8 | G | J | 99.5% and 0.5% | 0.5% and 99.5% |
| 9 | H | L | 99.5% and 0.5% | 0.5% and 99.5% |

In total, 18 such spiking samples were generated. The digestion of these spiking samples and subsequent analysis using an RP-LC coupled to an Orbitrap Fusion mass spectrometer was performed as described above for FIG. 3.

Across these 18 samples, a theoretical total of 140 positions is expected to differ by only one amino acid between the first and the second antibody within an otherwise identical sequence of ≥7 amino acids N- and C-terminally from that position, mimicking naturally occurring sequence variants. The resulting LC-MS/MS files were processed into protein data using the Software-Tool Byonic (Protein Metrics, San Carlos, CA/USA). This software compared experimental data (peptide masses (MS) and peptide fragments (MS/MS)) with theoretical data of the amino acid sequence of the respective “main antibody” (i.e. the antibody which is present in the sample at 99.5% percent) generated in silico. The results were ranked and visualized with the software Byologic (Protein Metrics), using the calculated dataset generated by Byonic. The protein data of the 18 samples which had been exported from Byologic were pooled and then analysed. By comparing the number of the correctly determined sequence variants in the spiked samples with the expected theoretical sequence variants, it was possible to assess the impact of individual filter settings on the overall sensitivity (true-positives) of the method in a quantitative way.

In FIGS. 4-8 and 10, in each case, the bars are presented in pairs with each pair containing a solid-white-filled bar on the left and a hatch-filled bar on the right. The height of each solid-white-filled bar represents sensitivity in % and corresponds to the scale given on the left vertical axis. Sensitivity is defined as the ratio of obtained true candidate sequence modifications to the calculated total of true candidate sequence modifications based on the sequences of the antibodies that were used for spiking. The height of each hatch-filled bar represents the total number of false positives and corresponds to the scale given on the right vertical axis. False positives are candidate sequence modifications that do not correspond to a true sequence modification resulting from the spiking and are given in absolute numbers. In FIGS. 4B and 5-8, the numbers for false positives represent the numbers of false positive candidate sequence modifications. In FIG. 4A the false positive numbers correspond to the numbers of peptide species with false positive sequence modifications, since the methods used to generate these results (unlike the method of the present disclosure) do not contain a step of pooling different peptide species with the same candidate modification into one hit.

FIGS. 4A and 4B are graphs depicting results of comparative experiments to demonstrate improved performance in comparison with alternative approaches. The graphs depict a pair of bars for each of six different configurations (respectively referred to as Settings A-F).

Settings A-C (shown in FIG. 4A) refer to configurations in which the methods of the present disclosure are not used. In each case, Trypsin is used as the main enzyme. In the configuration of Setting A, which may be referred to as “Trypsin_old”, Trypsin alone is used to obtain peptides for mass spectrometry analysis. In the configuration of Setting B, which may be referred to as “3Enzyme_old”, Trypsin was used first and Asp-N and Thermolysin were used as alternative enzymes to fill gaps in the sequence coverage where no tryptic “wild type” peptides were detectable. Only the most intense wild type peptide of an alternative enzyme was used to fill each gap. The configuration of Setting C, which may be referred to as “9Enzyme_old”, extends the approach of Setting B to use eight alternative enzymes/enzyme mixtures in addition to Trypsin to fill gaps in the sequence coverage where no tryptic “wild type” peptides were detectable. Only the most intense wild type peptide of an alternative enzyme was used to fill each gap. The eight alternative enzymes/mixtures were: Asp-N, Thermolysin, Chymotrypsin, Glu-C/Lys-C, Pronase, Pepsin, ProAlanase and Elastase. In Settings A-C, the sequence variants were filtered with an XIC Ratio >0.1%. Other filter settings were ppm=+/−4 ppm, size=750-3100 Dalton, accuracy score >140, intensity >1e7.

Settings D-F (Setting D is shown in FIG. 4A and Settings E and F are shown in FIG. 4B) refer to configurations in which nine enzymes/mixtures (Trypsin, Asp-N, Thermolysin, Chymotrypsin, Glu-C/Lys-C, Pronase, Pepsin, ProAlanase and Elastase) are used equally. All sequence variants corresponding to a wild type peptide retained after the filtering were used.

In the configuration of Setting D, which may be referred to as “9Enzymes_new”, all of the nine enzymes were used equally (Not only the most intense wild type peptide of an alternative enzyme was used to fill each gap) but no filtering according to embodiments of the present disclosure was applied. The “wild type” peptides were pre-filtered using the following pre-filter settings: ppm=+/−4 ppm, size=750-3100 Dalton, accuracy score >140, intensity >1e6.

In the configuration of Setting E, which may be referred to as “9Enzyme_new+filter1”, all nine enzymes were used along with filtering according to embodiments of the present disclosure. The filtering was performed with the following settings to ensure that no sequence variants were missed: SV Score >140, WT Score >140 ppm+/−4 ppm, MS1 Corr >0.95, PeptideLength=5-32 amino acids, WT Intensity >1e6, Exclude Cleavage Site=“yes”, Ratio Filter >2%, No_of_digests >=1, noPep >=2, XIC Ratio >0.1%.

In the configuration of Setting F, which may be referred to as “9Enzyme_new+filter2”, all nine enzymes were used along with filtering according to embodiments of the present disclosure. The filtering was performed with the following settings to achieve a similar sensitivity to the old approach (i.e. similar to Setting B) but with a reduction of false positives of >90%: SV Score >180, WT Score >260, ppm+/−3 ppm, MS1 Corr >0.96, PeptideLength=7-32 amino acids, WT Intensity >1e6, Exclude Cleavage Site=“yes”, Ratio Filter >10%, noPep >=2, No_of_digests >=1, XIC Ratio >0.1%.

It can be seen from FIGS. 4A and 4B that Setting D achieves high sensitivity but also high false positives, while Settings E and F demonstrate that embodiments of the present disclosure provide an improved balance of sensitivity to false positives. At Setting E, sensitivity is as high as for Setting D but with much fewer false positives. At Setting F, a sensitivity as high as any of the old approaches represented by Settings A-C is achieved but with much fewer false positives (>92% reduction).

FIG. 5A is a graph depicting results of further experiments to demonstrate how sensitivity and false positives vary for the method of embodiments of the present disclosure as a function of required coverage of amino acid sequence positions by different peptide species that each contain the modification and have each been derived from peptides obtained using different enzymatic digests (corresponding to the filter setting “No_of_digests”). Prior to the filtering step, the total number of predicted modifications (including true hits and false positive hits) was 25694. The first pair of bars, labelled “1”, corresponds to the case where a minimum coverage by a single peptide species is required for candidate selection. The second pair of bars, labelled “2”, corresponds to the case where a minimum coverage by two peptide species from two different digests is required for candidate selection. The third pair of bars, labelled “3”, corresponds to the case where a minimum coverage by three peptide species from three different digests is required for candidate selection. The fourth pair of columns, labelled “4”, corresponds to the case where a minimum coverage is by four peptide species from four different digest. The other filter settings were kept constant in each case and were as follows: SV Score >140, WT Score >140, ppm+/−4 ppm, MS1 Corr >0.95, PeptideLength=5-32 amino acids, WT Intensity >1e6, Exclude Cleavage Site=“yes”, Ratio Filter >0%, noPep >=1, XIC Ratio >0%.

It can be seen from FIG. 5A that increasing the minimum coverage by different peptide species from different enzymatic digests rapidly decreases the false positives while having only a limited negative effect on sensitivity. Requiring a minimum coverage of two peptides leads to a significant reduction in false positives with no measurable change in sensitivity. Requiring a minimum coverage of two peptides from two different digests provides a good balance of high sensitivity and few false positives.

FIG. 5B is a graph depicting results of further experiments to demonstrate how sensitivity and false positives vary for the method of embodiments of the present disclosure as a function of required coverage of amino acid sequence positions by different peptide species (corresponding to the filter setting “noPep”). Prior to the filtering step, the total number of predicted modifications (including true hits and false positive hits) was 25694. The first pair of bars, labelled “1”, corresponds to the case where a minimum coverage by a single peptide species is required for candidate selection. The second pair of bars, labelled “2”, corresponds to the case where a minimum coverage by two peptide species is required for candidate selection. The third pair of bars, labelled “3”, corresponds to the case where a minimum coverage by three peptide species is required for candidate selection. The fourth pair of columns, labelled “4”, corresponds to the case where a minimum coverage by four peptide species is required for candidate selection. The other filter settings were kept the same in each case and were as follows: SV Score >140, WT Score >140, ppm+/−4 ppm, MS1 Corr >0.95, PeptideLength=5-32 amino acids, WT Intensity >1e6, Exclude Cleavage Site=“yes”, Ratio Filter >0%, No_of_digests >=1, noPep >=1, XIC Ratio >0%.

It can be seen from FIG. 5B that increasing the minimum coverage by different peptide species rapidly decreases the false positives while having only a limited negative effect on sensitivity. Requiring a minimum coverage of two peptides leads to a significant reduction in false positives with no measurable change in sensitivity. Requiring a minimum coverage of three peptides provides a good balance of high sensitivity and few false positives.

FIG. 6 is a graph depicting results from further experiments to demonstrate how sensitivity and false positives vary for the method of embodiments of the present disclosure as a function of minimum values of the ratio of the number of peptide species covering the amino acid sequence position and containing the candidate sequence modification to the number of peptide species covering the amino acid sequence position and not containing the candidate sequence modification (corresponding to the filter setting “Ratio Filter”). The first pair of bars corresponds to the ratio value being required to be at least 1%. The second to fifth pairs of columns respectively represent minimum ratio values of 2%, 3%, 5% and 10%. The other filter settings were kept the same in each case and were as follows: SV Score >140, WT Score >140, ppm+/−4 ppm, MS1 Corr >0.95, PeptideLength=5-32 amino acids, WT Intensity >1e6, Exclude Cleavage Site=“yes”, No_of_digests >=1, noPep >=2, XIC Ratio >0.1%.

It can be seen from FIG. 6 that increasing the minimum ratio value leads to a rapid decrease in the false positives with a significantly slower reduction in the sensitivity. As in FIGS. 5A and 5B, the total number of predicted modifications (including true hits and false positive hits) prior to the filtering step here was 25694. Increasing the minimum ratio value from 1% to 2% significantly decreases false positives while not significantly affecting sensitivity. Increasing the minimum ratio value to 3% leads to a good balance between high sensitivity and very few false positives. Higher minimum ratio values lead to extremely few false positives while keeping sensitivity at useful levels.

FIG. 7 is a graph depicting results from further experiments to demonstrate how sensitivity and false positives vary for the method of embodiments of the present disclosure as a function of required minimum intensities of mass spectrometry measurements (corresponding to the filter setting “WT Intensity”). Pairs of bars are shown corresponding respectively to a minimum intensity that increases from left to right from 1e+05 to 1e+08. The other filter settings were kept the same in each case and were as follows (with no Ratio Filter): SV Score >140, WT Score >140, ppm+/−4 ppm, MS1 Corr >0.95, PeptideLength=5-32 amino acids, Exclude Cleavage Site=“yes”, Ratio Filter >0%, No_of_digests >=1, noPep >=2, XIC Ratio >0.1%.

It can be seen from FIG. 7 that increasing the minimum intensity value leads to a rapid decrease in the false positives with a significantly slower reduction in the sensitivity. As in FIGS. 5 and 6, the total number of predicted modifications (including true hits and false positive hits) prior to the filtering step here was 25694. Increasing the minimum intensity value from 1e+05 to 1e+06 significantly decreases false positives while not significantly affecting sensitivity. Increasing the minimum intensity value to 1e+07 leads to a good balance between high sensitivity and very few false positives. Higher minimum intensity values lead to extremely few false positives while keeping sensitivity at useful levels.

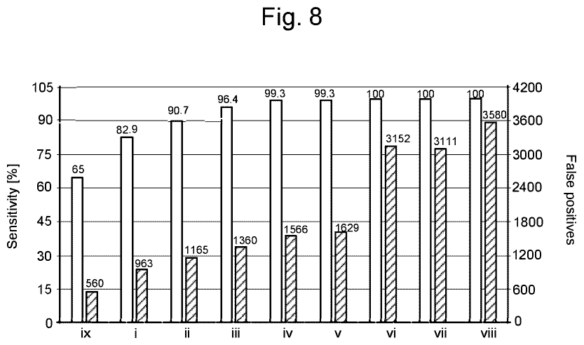

FIG. 8 is a graph depicting results from further experiments to demonstrate how sensitivity and false positives vary for the method of embodiments of the present disclosure as a function of increasing numbers of enzymes. Pairs of bars are shown corresponding respectively to enzyme groups containing 2-9 enzymes as follows:
Group i=Trypsin, Pepsin;
Group ii=Trypsin, Pepsin, ProAlanase;
Group iii=Trypsin, Thermolysin, Pepsin, ProAlanase;
Group iv=Trypsin, Thermolysin, Pronase, Pepsin, ProAlanase;
Group v=Trypsin, Thermolysin, AspN, Pronase, Pepsin, ProAlanase;
Group vi=Trypsin, Thermolysin, AspN, Elastase, Pronase, Pepsin, ProAlanase;
Group vii=Trypsin, Thermolysin, AspN, Elastase, GluC, Pronase, Pepsin; ProAlanase;
Group viii=Trypsin, Thermolysin, AspN, Elastase, Chymotrypsin, GluC, Pronase, Pepsin, ProAlanase.

As a control, the sensitivity and false positives for only one enzyme (Pepsin) are shown in Group ix. Out of nine used enzymatic digests, Pepsin showed the highest sensitivity, when using the below mentioned filter settings. Therefore Pepsin instead of Trypsin, the gold standard used for enzymatic digests, was used here as comparison.

The same filter settings were applied in each case and were as follows: SV Score >140, WT Score >140, ppm+/−4 ppm, MS1 Corr >0.95, PeptideLength=5-32 amino acids, WT Intensity >1e6, Exclude Cleavage Site=“yes”, Ratio Filter >2%, No_of_digests >=1, noPep >=2, XIC Ratio >0.1%.

It can be seen from FIG. 8 that increasing the number of enzymes increases sensitivity but also the false positives. As in FIGS. 5 to 7, the total number of predicted modifications (including true hits and false positive hits) prior to the filtering step here was 25694, when using all nine enzymes. A much better sensitivity can already be achieved by using two enzymes, compared to the control where only data from a single digest were used. A particularly good balance of high sensitivity to low numbers of false positives is achieved for 5-6 enzymes, although higher numbers of enzymes achieve even better sensitivity with manageable false positives.

In some embodiments, the method further comprises identifying one or more groups of peptide species in the received protein data, wherein each group of peptide species exclusively contain peptide species that all have the same candidate sequence modification. The candidate sequence modification is in the determined subset of candidate sequence modifications and different for each group. Thus, the grouping process may be performed after any combination of the filtering steps described above. The method may comprise outputting data representing which peptide species are in each of the identified groups. The data may comprise a list of the peptide species in each identified group. The data may be adapted for display as a graph or other non-text based representation of the groups. The methodology may be implemented by providing selectable options to define the one or more groups. The selectable options may include a definition of filter settings to be applied (e.g., corresponding WT Intensity >1e6, SV Score >140, Peptide Length 5-32, MS1 Score >0.95, etc.). The selectable options may include a definition of the modification (e.g., Alanine->Serine SV exchange). The selectable options may include a definition of a location of the modification. The location may include definition of either or both of a chain (e.g., light chain, LC) and an amino acid (e.g., amino acid 25).

In some embodiments, the determination of the subset in step S7 comprises selecting candidate sequence modifications dependent on the candidate sequence modifications satisfying a quantification condition. The quantification condition broadly requires that a detected relative amount of peptide species having the candidate sequence modification (i.e., relative to a total detected amount of the corresponding peptide species with and without the modification) should be relatively high. The inventors reasoned that satisfaction of such a quantification condition is likely to be strongly correlated with the candidate sequence modification being a true sequence modification, and therefore provide an effective basis for filtering. A filter setting corresponding to this type of filtering may be referred to as “Quant” herein.

A challenge with implementing such a filter effectively is formulating an appropriate metric to accurately represent the detected relative amount of peptide species having the candidate sequence modification. The complexity of the situation is illustrated schematically in FIG. 9, which depicts example results from mass spectrometry measurements.

FIG. 9 schematically depicts as boxes peptides species having and not having a candidate sequence modification. The right column of boxes represents peptide species having the candidate modification (schematically indicated by the black vertical bar in each box in the right column). The left column of boxes represents corresponding peptide species not having the candidate modification (i.e., wild type peptide species). Nine rows (a)-(i) are presented with each row containing a pair of corresponding peptide species (without and with the candidate sequence modification). Within each box, a charge state is indicated by z2 (doubly charged), z3 (triply charged) or z4 (quadruply charged). The area under a portion of the curve of intensity against time in the mass spectrometry measurements that corresponds to the respective peptide species is indicated after “Area:” with units of counts*s. Boxes marked “n.d.” correspond to peptide species that were not detected (with the modification). The rows correspond to different peptide species or different charge states of the peptide species. In the example shown, the peptide species are from three different digests (indicated Dig 1, Dig 2, and Dig 3). For each of Dig 1 and Dig 2, a single peptide species is shown (labelled Pep 1 for Dig 1 and Pep 2 for Dig 2) in two different charge states (z2 and z3). Rows (a) and (b) correspond to Pep 1 from Dig 1 and rows (c) and (d) correspond to Pep 2 from Dig 2. For Dig. 3, two peptide species are shown (labelled Pep 3 and Pep 4). Rows (e) and (f) correspond to Pep 3, with charge states of z2 and z3. Rows (g), (h) and (i) correspond to Pep 4, with charge states of z2, z3 and z4. In this particular example, Dig 1 was a Chymotrypsin digest and Pep 1 was the peptide species corresponding to amino acid sequence range 613-629. Dig 2 was a Trypsin digest and Pep 2 was the peptide species corresponding to amino acid sequence range 608-623. Dig 3 was a LysC+GluC digest, Pep 3 was the peptide species corresponding to amino acid sequence range 604-620, and Pep 4 was the peptide species corresponding to amino acid sequence range 604-623 deriving from the same LysC+GluC digest.

Various metrics could in principle be formulated to represent the detected relative amount of peptide species having the sequence modification.

Examples of metrics considered by the inventors are described below and referred to as Metrics 1-7. The determined value of each metric may be compared with a predetermined quantification threshold to implement the Quant filtering (i.e., to determine whether or not to include the candidate sequence modification in the subset in step S7).

In an example arrangement, the mass spectrometry measurements, which may be liquid chromatography-tandem mass spectrometry (LC-MS/MS), output curves of intensity against time. For a given peptide species, a detected amount of the peptide species is strongly correlated both with a maximum (peak intensity) of a portion of the curve that corresponds to the peptide species and with an area under the portion of the curve that corresponds to the peptide species. In the discussion of the metrics below, reference is made to the “area” when discussing detected amounts of individual peptide species. It will be understood that the methodology could also be implemented by using the maximum (peak intensity) instead of the area or, indeed, any other suitable parameter extracted from the mass spectrometry measurements that correlates with the detected amount of the peptide species.

The percentages listed in column 21 in FIG. 9 represent, for each row, the ratio of the area of the peptide species in the right column (i.e., with the modification) to the sum of the areas of the peptide species in the right and left columns (i.e., with and without the modification), expressed as a percentage. The percentages in column 21 thus provide information relevant to determining the detected relative amount of peptide species having the candidate sequence modification. However, it can be seen that the percentages vary significantly in size. The percentages listed in column 22 represent, for each group of rows corresponding to a given peptide species (including all charge states), the ratio (expressed as a percentage) of the sum of the areas of all charge states with the modification (i.e., the areas of all of the boxes in the right column for the peptide species being considered) to the sum of the areas of all charges states for that peptide species with and without the modification (i.e., the sum of the areas of all boxes in the right column and the left column for the peptide species being considered). Thus, for Pep 1 for example, the value 0.15% results from 100%×4.3×10⁶/(4.3×10⁶+4.4×10⁸+2.4×10⁹). Again significant variation is seen in the percentages in column 22.

In Metrics 1 and 3, only the most intense (largest area) of all wildtypes corresponding to peptide species that carry the modification is used to calculate the relative amount of the modification. Thus, the metrics are calculated using just one of the rows in FIG. 9 (i.e., one of the values in column 21). In the case of Metric 1, the selection is limited to peptide species from a selected one of the digests. Typically, the selected digest would be a Trypsin digest but this is not essential. In the case of Metric 3, peptide species from all digests are considered.

In an example, the selected digest for Metric 1 may be Dig 2 (Trypsin). One peptide species (Pep 2) with two charge states was derived using Dig 2 in the example of FIG. 9. According to Metric 1, the largest area wildtype corresponding to a peptide species that carries the modification for Pep 2 is row (c) because the peptide species with the modification is not detected for the charge state corresponding to row (d) (i.e., the box in the right column is “n.d.”). Metric 1 would thus be 0.33% in this example.

If the selected digest for Metric 1 was Dig 3 instead of Dig 2, then the largest area wildtype corresponding to a peptide species that carries the modification would be row (f), corresponding to the z3 charge state of Pep 3. Metric 1 would thus be 0.13% in this example.

For Metric 3, peptide species from all digests are considered so the largest area wildtype corresponding to a peptide species that carries the modification used to calculate Metric 3 would also be row (f) because row (f) has the largest area wildtype corresponding to a peptide species that carries the modification for all of the digests. Row (b) has a larger area wildtype but the version with the modification (right column) is not detected (“n.d.”), so row (b) is not used.

Metric 2 is a variation of Metrics 1 and 3 in which all charge states of peptide species having the modification are considered, regardless of whether the individual charge states have the modification. Metric 2 is then calculated according to the methodology described above for column 22. Metric 2 is the percentage in column 22 that corresponds to the peptide species that contains the largest area wildtype (considered over all charge states). Metric 2 may consider only peptide species from a selected digest or peptide species from all digests. If the selected digest is Dig 2, the output from Metric 2 would be 0.07% because only one peptide species is derived from Dig 2. If the selected digest is Dig 3, the output from Metric 2 would be 0.30% because Pep 3 is the peptide species derived using Dig 3 that contains the largest area wildtype. If all digests are considered, the output from Metric 2 would be 0.15% because Pep 1 has the largest area wildtype overall (row (b)).

In Metrics 4-7, metrics are calculated based on combining information about areas of peptide species from multiple different digests.

Metric 4 considers all combinations of peptide species and charge states that have the modification (i.e., all rows in FIG. 9 for which the right column is not “n.d.”), including peptide species from multiple (e.g., all) digests, but uses pre-filtering to filter out rows where the area of the wildtype peptide species (left column) is below a threshold (e.g., 10⁷ counts*s). Thus, only rows in which the left column area is greater than the threshold and the right column is not “n.d.” are considered. The metric is then calculated as a mean of the corresponding percentages in column 21. Thus, if the threshold area was 10⁷ counts*s, in the example of FIG. 9 all rows not having “n.d.” in the right column would be considered. Metric 4 would then be the mean of the values in column 21 for rows (a), (c), (e), (f), and (h), which would result in 0.66%. If the threshold area was raised 3×10⁸ counts*s, then row (e) would be filtered out and Metric 4 would then instead be the mean of the values in column 21 for rows (a), (c), (f), and (h), which would result in 0.52%.

In Metrics 5-7, a methodology which is referred to herein as weighted quantification is used. In this type of approach, information about areas may be combined to obtain a quantification metric expressed as a percentage according to the following formula:

$$Metric = \frac{\sum area of modified}{\sum area of modified + \sum area of wildtype} \times 100$$

where Σ area of modified is the sum of the areas of the modified peptide species and Σ area of wildtype is the sum of the areas of wildtype peptide species corresponding to the modified peptide species (with the correspondence requiring also a correspondence in the charge state). In each case, only charge states for which there is a detected peptide species with the modification are considered. Thus, in the example of FIG. 9, rows (b), (d), (g) and (i) would not contribute.

In Metric 5, peptide species from all digests are taken into account. Thus, in the example of FIG. 9, the output from Metric 5 would take into account rows (a), (c), (e), (f) and (h). The output of the Metric 5 is the sum of all of the areas in the right column of rows (a), (c), (e), (f) and (h) divided by the sum of all of the areas in both columns of rows (a), (c), (e), (f) and (h), which equals 0.47%.

In Metric 6, only peptide species from sequence-specific enzymes are considered. In the example of FIG. 9, this results in exclusion of peptide species from Dig 1, which was performed using Chymotrypsin. The output of Metric 6 is the sum of all of the areas in the right column of rows (c), (e), (f) and (h) divided by the sum of all of the areas in both columns of rows (c), (e), (f) and (h), which equals 0.39%.

In Metric 7, a combination of the approaches of Metrics 5 and 6 is used to provide fuller coverage. Thus, only sequence-specific enzymatic digests are used where there is coverage by these sequence-specific enzymatic digests, and gaps are filled by non-specific enzymatic digests. In other words, for candidate modifications where sequence-specific enzymatic digests provide coverage, the sequence-specific enzymatic digests are used as described above for Metric 6. In cases where the modification is not covered by any sequence-specific enzymatic digests, it is necessary to use the non-specific enzymatic digests, which means all peptide species which are available for this candidate modification and position are used, as described above for Metric 5. That case is special, because only non-specific enzymatic digests are used. Metric 5 can use sequence-specific and non-specific enzymatic digests.

The efficacy of the various metrics was tested using samples spiked to contain 140 sequence variations at a proportion of 1%. The results are shown in Table 1 below. The selected digest for Metrics 1 and 2 was Trypsin. The threshold for Metric 4 was set at 10⁷ counts*s.

TABLE 1

| Metric | 1 | 2 | 3 | 4 | 5 | 6 | 7 |
|---|---|---|---|---|---|---|---|
| SVs (%) | 81 | 81 | 100 | 100 | 100 | 91 | 100 |
| 5x too high | 7 | 8 | 17 | 0 | 0 | 1 | 1 |
| 5x too low | 3 | 3 | 2 | 18 | 8 | 3 | 3 |
| mean | 4.3 | 8.2 | 19.4 | 2.8 | 2.3 | 2.3 | 2.2 |
| deviation | | | | | | | |

“SVs(%)” represents the percentage of true sequence variations detected (100% = 140 SVs). It is desirable for this value to be as high as possible.
“5x too high” represents the number of sequence variations quantified at a level that is greater than 5 times higher than 1% (i.e., >5%). It is desirable for this value to be as low as possible.
“5x too low” represents the number of sequence variations quantified at a level that is equal to or less than 5 times lower than 1% (i.e., <0.2%). It is desirable for this value to be as low as possible.
“mean deviation” represents the absolute mean deviation from the 1% target of all quantifications for the SVs which can be quantified by the method. It is desirable for this value to be as low as possible.

It can be seen that the best performing approaches are those based on metrics 5, 6 and 7, which all achieve very low mean deviations from the target of 1%. Metric 6 achieves better performance than Metric 5 in respect of “5× too low” but is worse with respect to “SVs(%)”. Metric 7 achieves the best overall performance.

Based on the above insights, in an arrangement, the quantification condition is configured to indicate that an amount detected by the mass spectrometry measurements of at least a selected subset of peptide species with the candidate sequence modification relative to a total amount detected by the mass spectrometry measurements of the same peptide species (which may be plural peptide species where the subset comprises a plurality of peptide species) with and without the candidate sequence modification is above a predetermined quantification threshold. In an arrangement, the selected subset comprises a plurality or all peptide species from a plurality or all of the at least two different enzymatic digests used (e.g., as represented by Metric 5, 6 or 7 discussed above). The quantification condition may thus use the expression

$$Metric = \frac{\sum area of modified}{\sum area of modified + \sum area of wildtype} \times 100$$

mentioned above, or similar or equivalent, such as a value proportional thereto, to calculate a metric to compare to the predetermined quantification threshold.

In some arrangements, the at least two different enzymatic digests comprise one or more sequence-specific enzymatic digests and one or more non-specific enzymatic digests. This was the case in the example illustrated in FIG. 9. In such arrangements, the selected subset of the peptide species may be selected to exclude peptide species derived using the one or more non-specific enzymatic digests, as was the case in Metric 6. The selected subset may thus consist of peptide species from plural sequence-specific enzymatic digests. The selected subset of peptide species may be selected to include peptide species derived using the one or more non-specific enzymatic digests for candidate sequence modifications that are not covered by at least one peptide species derived using a sequence-specific enzymatic digests. Thus, gaps without sequence-specific enzymatic digest coverage may be filled using non-specific enzymatic digests (e.g., Metric 7). Alternatively, all the relevant peptide species may be included, with no selection of a subset of peptide species based on the nature of the enzymatic digest used (e.g., Metric 5).

Sequence-specific enzymatic digests within the meaning of the present disclosure may be digests performed with at least one proteolytic enzyme (protease) that cleaves the protein N-terminally or C-terminally of a specific amino acid or sequence of adjacent amino acids in the sequence of the protein in a predictable way, e.g. trypsin cleaves a protein C-terminally of the amino acid K or R in the amino acid sequence of a protein. Other enzymatic digests, i.e., enzymatic digests that are not sequence-specific, may be referred to in the present disclosure as non-specific enzymatic digests. The cleavage sites created when using non-specific enzymatic digests may be less predictable or unpredictable, but are reproducible for the digest of a specific protein using a specific protease.

In an arrangement, the sequence-specific enzymatic digests include or consist of one or more of the following enzymes: Trypsin, Endoproteinase AspN, Endoproteinase LysC, Endoproteinase GluC.

In an arrangement, the non-specific enzymatic digests include or consist of one or more of the following enzymes: Thermolysin, Elastase, Pronase, ProAlanase, Pepsin, Chymotrypsin.

FIG. 10 is a graph depicting results from further experiments to demonstrate how sensitivity and false positives vary for the method of embodiments of the present disclosure as a function of an increasing quantification threshold (corresponding to the filter setting “Quant” and using Metric 7 described above). Pairs of bars are shown corresponding respectively to quantification thresholds (Quant) of 0, 0.1, 0.2, 0.3 and 0.5, increasing from left to right. Increasing the quantification threshold will improve suppression of false positives but may also reduce sensitivity. The quantification threshold can be selected according to requirements and the above values are exemplary only. In some embodiments, a quantification threshold is selected to be equal to or greater than 0.05, 0.1, 0.2, or 0.3. In some embodiments, the quantification threshold is additionally (where applicable) or alternatively selected to be equal to or less than 0.6, 0.5, 0.4, 0.3, 0.2 or 0.1. The other filter settings were kept the same in each case and were as follows: SV Score >140, WT Score >140, ppm+/−4 ppm, MS1 Corr >0.95, PeptideLength=5-32 amino acids, WT Intensity >1e6, Exclude Cleavage Site=“yes”, Ratio Filter >2%, noPep >=2. Selection of hits for each candidate modification (defined by the position in the protein and the type of the modification) was performed using only the modified peptide species resulting from sequence-specific enzymatic digests (i.e., Asp-N, Trypsin and GluC+LysC in this case). If no specific peptides are available, all hits from unspecific enzymatic digests are considered for quantification.

It can be seen from FIG. 10 that increasing the Quant filter setting leads to a rapid decrease in the false positives with a tolerably slow reduction in the sensitivity. With a Quant filter setting of 0.1, for example, the number of false positives was reduced by a valuable 33% (from 3586 to 2405) for a reduction in obtained true positives of only 1.4% (from 100.0% to 98.6%). Note that the result obtained for a Quant filter setting=0 (no Quant filter) corresponds already to the result of applying the filters described above with reference to FIGS. 5B, 6 and 7:
at least 2 peptides per modification (FIG. 5B)
Ratio Filter >2% (FIG. 6)
Only high intensity WT (>1e6) (FIG. 7).

Due to this filtering, the number of false positives is already highly reduced, even prior to applying the Quant filter. In this context, the observed improvement of 33% is especially significant.

1. A computer-implemented method of extracting information about protein sequence modifications in a protein, comprising:
receiving protein data derived at least partially from mass spectrometry measurements performed on peptides obtained by at least two different enzymatic digests performed on respective sub-samples of a representative sample of a protein;
identifying candidate sequence modifications in the protein using the received protein data;
determining a subset of the candidate sequence modifications that have a higher average probability of representing true sequence modifications than the rest of the candidate sequence modifications; and
outputting data representing the determined subset of candidate sequence modifications, wherein:
the determination of the subset of candidate sequence modifications comprises a step of selecting candidate sequence modifications dependent on the candidate sequence modifications being at amino acid sequence positions that are covered by at least two different peptide species that each contain the modification.

2. The method of claim 1, wherein each peptide species is defined by at least the following:
i) an amino acid sequence;
ii) the enzymatic digest that produced the peptide species; and
iii) a modification status indicating whether a candidate modification is present and, if a candidate modification is present, the nature and amino acid sequence position of the modification.

3. The method of claim 2, wherein each peptide species is further defined by:
iv) a charge state in the mass spectrometry measurements.

4. The method of claim 1, comprising:
pre-processing the protein data to identify data relating to a selected subset of peptide species and excluding the selected subset of peptide species from use in the determination of the subset of candidate sequence modifications.

5. The method of claim 4, wherein the pre-processing comprises excluding peptide species for which a) a candidate modification is present; and b) a highest intensity in the mass spectrometry measurements of a corresponding peptide species with the same amino acid sequence but without the candidate modification is below a predetermined intensity threshold.

6. The method of claim 4, wherein the pre-processing comprises excluding peptide species for which a) a candidate modification is present; and b) a highest accuracy score in the mass spectrometry measurements of a corresponding peptide species with the same amino acid sequence but without the candidate modification is below a predetermined score threshold, the accuracy score representing a degree of matching between theoretical and observed fragments in the mass spectrometry measurements.

7. The method of claim 4, wherein the pre-processing comprises:
excluding each peptide species having a candidate modification at a cleavage site for the enzymatic digest that produced the peptide species; and/or
excluding peptide species having a length above a predetermined length threshold.

8. The method of claim 1, wherein the determination of the subset of candidate sequence modifications comprises a step of selecting candidate sequence modifications dependent on the candidate sequence modifications being at amino acid sequence positions that are covered by at least two different peptide species that each contain the modification and have been derived from peptides obtained using different enzymatic digests.

9. The method of claim 1, wherein the determination of the subset of candidate sequence modifications comprises a step of selecting candidate sequence modifications dependent on the candidate sequence modifications being at amino acid sequence positions where a ratio of the number of peptide species covering the amino acid sequence position and containing the candidate sequence modification to the number of peptide species covering the amino acid sequence position and not containing the candidate sequence modification is equal to or higher than a predetermined ratio threshold.

10. A computer-implemented method of extracting information about protein sequence modifications in a protein, comprising:
receiving protein data derived at least partially from mass spectrometry measurements performed on peptides obtained by at least two different enzymatic digests performed on respective sub-samples of a representative sample of a protein;
identifying candidate sequence modifications in the protein using the received protein data;
determining a subset of the candidate sequence modifications that have a higher average probability of representing true sequence modifications than the rest of the candidate sequence modifications; and
outputting data representing the determined subset of candidate sequence modifications, wherein:
the determination of the subset of candidate sequence modifications comprises a step of selecting candidate sequence modifications dependent on the candidate sequence modifications being at amino acid sequence positions where a ratio of the number of peptide species covering the amino acid sequence position and containing the candidate sequence modification to the number of peptide species covering the amino acid sequence position and not containing the candidate sequence modification is equal to or higher than a predetermined ratio threshold.

11. The method of claim 10, wherein the predetermined ratio threshold is in the range of 2-10%.

12. The method of claim 1, wherein the determination of the subset of candidate sequence modifications comprises a step of selecting candidate sequence modifications dependent on the candidate sequence modifications satisfying a quantification condition, the quantification condition indicating that an amount detected by the mass spectrometry measurements of at least a selected subset of peptide species with the candidate sequence modification relative to a total amount detected by the mass spectrometry measurements of the same peptide species with and without the candidate sequence modification is above a predetermined quantification threshold.

13. A computer-implemented method of extracting information about protein sequence modifications in a protein, comprising:
receiving protein data derived at least partially from mass spectrometry measurements performed on peptides obtained by at least two different enzymatic digests performed on respective sub-samples of a representative sample of a protein;
identifying candidate sequence modifications in the protein using the received protein data;
determining a subset of the candidate sequence modifications that have a higher average probability of representing true sequence modifications than the rest of the candidate sequence modifications; and
outputting data representing the determined subset of candidate sequence modifications, wherein:
the determination of the subset of candidate sequence modifications comprises a step of selecting candidate sequence modifications dependent on the candidate sequence modifications satisfying a quantification condition, the quantification condition indicating that an amount detected by the mass spectrometry measurements of at least a selected subset of peptide species with the candidate sequence modification relative to a total amount detected by the mass spectrometry measurements of the same peptide species with and without the candidate sequence modification is above a predetermined quantification threshold.

14. The method of claim 13, wherein the selected subset comprises a plurality or all peptide species from a plurality or all of the at least two different enzymatic digests.

15. The method of claim 13, wherein the at least two different enzymatic digests comprise one or more sequence-specific enzymatic digests and one or more non-specific enzymatic digests.

16. The method of claim 15, wherein the selected subset of the peptide species is selected to exclude peptide species derived using the one or more non-specific enzymatic digests, at least for candidate sequence modifications that are covered by at least one peptide species derived using a sequence-specific enzymatic digest.

17. The method of claim 16, wherein the selected subset of peptide species is selected to include peptide species derived using the one or more non-specific enzymatic digests for candidate sequence modifications that are not covered by at least one peptide species derived using a sequence-specific enzymatic digest.

18. The method of claim 16, wherein the sequence-specific enzymatic digests include one or more of the following: Trypsin, Endoproteinase AspN, Endoproteinase LysC, Endoproteinase GluC.

19. The method of claim 16, wherein the non-specific enzymatic digests include one or more of the following: Thermolysin, Elastase, Pronase, ProAlanase, Pepsin, Chymotrypsin.

20. The method of claim 13, wherein the mass spectrometry measurements comprise liquid chromatography-tandem mass spectrometry.

21. The method of claim 20, wherein for each peptide species the amount detected by the mass spectrometry measurements is defined as:
an area under a portion of a curve of intensity against time in the mass spectrometry measurements that corresponds to the peptide species; or
a maximum in a portion of a curve of intensity against time in the mass spectrometry measurements that corresponds to the peptide species.

22. The method of claim 1, wherein each enzymatic digest uses a different one or combination of the following: Trypsin; Thermolysin; AspN; Elastase; Chymotrypsin; LysC; LysN; GluC; ArgC; Pronase; Pepsin; ProAlanase.

23. The method of claim 1, wherein the received protein data is derived from mass spectrometry measurements performed on peptides obtained by five or six different enzymatic digests performed on respective sub-samples of the representative sample of the protein, preferably wherein each of the five or six enzymatic digests uses a different one of the following: Trypsin, Thermolysin, AspN, Pronase, Pepsin, ProAlanase.

24. The method of claim 1, further comprising:
identifying one or more groups of peptide species in the received protein data, each group of peptide species exclusively containing peptide species that all have the same candidate sequence modification, the candidate sequence modification being in the determined subset of candidate sequence modifications and different for each group; and
outputting data representing which peptide species are in each of the identified groups.

25. A computer-readable medium carrying the computer program, the computer program comprising instructions which, when the program is executed by a computer, cause the computer to carry out the method of claim 1.
